(19) United States
(12) Patent Application Publication
(10) Pub. No.: US 2024/0382557 A1
(43) Pub. Date: Nov. 21, 2024

(54) INHIBITION OF TUMOR NECROSIS FACTOR, PRO-INFLAMMATORY CYTOKINES AND OTHER INFLAMMATORY RESPONSE MEDIATORS

(71) Applicant: Allegro Pharmaceuticals, LLC, San Juan Capistrano, CA (US)

(72) Inventors: Hampar L. Karageozian, San Juan Capistrano, CA (US); John Y. Park, Santa Ana, CA (US); Vicken H. Karageozian, San Juan Capistrano, CA (US); Lisa S. Karageozian, San Juan Capistrano, CA (US)

(21) Appl. No.: 18/618,282

(22) Filed: Mar. 27, 2024

Related U.S. Application Data
(63) Continuation of application No. 17/707,332, filed on Mar. 29, 2022, now abandoned.
(60) Provisional application No. 63/167,879, filed on Mar. 30, 2021.

(51) Int. Cl. A61K 38/08; A61P 37/06

(52) U.S. Cl. CPC A61K 38/08; A61P 37/06

(57) ABSTRACT

Peptide compositions and their use for inhibiting tumor necrosis factor, pro-inflammatory cytokines and other inflammatory response mediators.

### RELATED APPLICATION

This patent application is a continuation of co-pending U.S. patent application Ser. No. 17/707,332 entitled Inhibition of Tumor Necrosis Factor, Pro-Inflammatory Cytokines and Other Inflammatory Response Mediators filed Mar. 29, 2022, which claims priority to U.S. Provisional Patent Application No. 63/167,879 entitled Inhibition of Tumor Necrosis Factor, Pro-Inflammatory Cytokines and Other Inflammatory Response Mediators filed Mar. 30, 2021, the entire disclosure of each such application being expressly incorporated herein by reference.

### FIELD OF THE INVENTION

This disclosure relates generally to the fields of chemistry, life sciences, pharmacy and medicine and more particularly to pharmaceutical preparations and their use in the treatment disease.

### BACKGROUND

Cytokines are proteins, peptides or glycoproteins produced in the body by lymphocytes and monocytes. A number of cytokine families have been identified, including chemokines, interferons, interlukens (IL), monokines, lymphokines, tumor necrosis factor and erytheropoietin,

Cytokines perform a range of functions, including the triggering and regulating inflammatory responses. Some cytokines are pro-inflammatory while others are anti-inflammatory. Pro-inflammatory cytokines are produced by activated macrophages which are responding to the presence of aberrant cells, foreign substances or pathological invaders such as certain bacteria and viruses.

When large amounts of pro-inflammatory cytokines are produced, a condition known as cytokine storm (also referred to as hypercytokinemia or cytokine release syndrome) can result. Such cytokine storms have been reported to occur in patients suffering from disorders such as graft-versus-host-disease, pancreatitis, cancers, influenzas, mycobacterial infections and viral infections including, most recently, SARS-CoV-2 (also known as COVID-19). A cytokine storm can result in serious damage to cells, tissues and organs and, in severe cases organ failure and death.

Pro-inflammatory and anti-inflammatory cytokines are also involved in the pathogenesis of various autoimmune and other disorders, such as rheumatoid arthritis (RA), osteoarthritis, juvenile arthritis, psoriatic arthritis, plaque psoriasis, ankylosing spondylitis, ulcerative colitis (UC), and Crohn's disease.

Also, chronic pro-inflammatory conditions are associated with many ocular diseases, including dry eye disease, age related macular degeneration and diabetic retinopathy. During this process, tissue resident and peripheral immune cells are activated and contribute to formation of a cytotoxic inflammatory state that causes tissue damage and vision loss.

There exists a need for the development of new therapies to prevent or lessen the effects of pro-inflammatory cytokines in human or animal subjects.

### SUMMARY

The present disclosure describes peptide compositions comprising Risuteganib (ALG-1001) or other effective peptides and their uses and associated methods for inhibiting tumor necrosis factor, pro-inflammatory cytokines and other inflammatory response mediators.

In accordance with the present disclosure, compositions comprising Risuteganib (ALG-1001) or other effective peptides are useable for preventing or reducing severity of hypercytokinemia, cytokine release syndrome or cytokine storm in a subject in need thereof.

Further in accordance with the present disclosure, compositions comprising Risuteganib (ALG-1001) or other effective peptides are useable for inhibiting one or more endogenous substance selected from: tumor necrosis factor (TNF), interluken 6 (IL6), CCL2, ITGAX, CSF1, and SRC, in a in a subject in need thereof.

Further in accordance with the present disclosure, compositions comprising Risuteganib (ALG-1001) or other effective peptides are useable to lessen or otherwise treat inflammatory responses to infectious diseases such as, for example, influenza, mycobacterial infection, viral infection, coronavirus infection, SARS-COV-2 (COVID-19) infection or pneumonia.

Further in accordance with the present disclosure, compositions comprising Risuteganib (ALG-1001) or other effective peptides are useable to deter or treat autoimmune disorders such as, for example, rheumatoid arthritis (RA), osteoarthritis, juvenile arthritis, psoriatic arthritis, plaque psoriasis, ankylosing spondylitis, ulcerative colitis (UC), and Crohn's disease.

Further in accordance with the present disclosure, compositions comprising Risuteganib (ALG-1001) or other effective peptides are useable to mitigate inflammatory or cytokine-mediated tissue impairment or damage in a human or non-human animal subject who has been diagnosed with a disease or disorder selected from; an influenza, mycobacterial infection, viral infection, coronavirus infection, SARS-COV-2 (COVID-19) infection, pneumonia, an autoimmune disorder, rheumatoid arthritis (RA), osteoarthritis, juvenile arthritis, psoriatic arthritis, plaque psoriasis, ankylosing spondylitis, ulcerative colitis (UC), or Crohn's disease.

As used herein, the term “patient or “subject” refers to either a human or non-human animals, such as humans, primates, mammals, and vertebrates.

As used herein, the term “treat” or “treating” or “treatment” refers to preventing, eliminating, curing, deterring, reducing the severity or reducing at least one symptom of a condition, disease or disorder.

As used herein, the phrase “effective amount” or “amount effective to” refers to an amount of an agent that produces some desired effect at a reasonable benefit/risk ratio. In certain embodiments, the term refers to that amount necessary or sufficient to bring about the specified treatment or other effect. The effective amount may vary depending on such factors as the disease or condition being treated, the particular composition being administered, or the severity of the disease or condition. Persons of skill in the art may empirically determine the effective amount of a particular agent without necessitating undue experimentation.

### BRIEF DESCRIPTION OF THE DRAWINGS

The following figures are included in this patent application and referenced in the following Detailed Description. These figures illustrate certain aspects or embodiments of the present disclosure but do not limit the scope of the present disclosure in any way:

FIG. 1 is a TNF-A pathway diagram showing Risuteganib-Induced Expression Changes in LOG2-Fold-Change scale.

FIG. 2 is a bar graph showing normalized gene expression (TPM) for TNF, IL6, CCL2, ITGAX, CSF1 and SRC genes following treatment with risuteganib (ALG-1001) or control.

FIG. 3 s a graphic representation showing biological processes which are down-regulated by risuteganib.

FIG. 4 is a bar graph showing biological pathways enriched with genes that are down-regulated by risuteganib.

FIG. 5 is a bar graph showing the effects of risuteganib, pro-inflammatory cytokines, risuteganib+pro-inflammatory cytokines and control on gene expression of CCL2, CCL20, CXCL5, CXCL6, TNFAIP6, TNFRSF11B and SELE.

FIG. 6 is a bar graph showing the effects of risuteganib, pro-inflammatory cytokines, risuteganib+pro-inflammatory cytokines and control on gene expression of CFH, IL6, IL7R and TNFAIP3.

APPENDIX A is a table showing expression levels of one hundred and thirty-five (135) genes in primary human immune cells following treatment with either a) Risuteganib or b) Control.

### DETAILED DESCRIPTION

The following detailed description and the accompanying drawings to which it refers are intended to describe some, but not necessarily all, examples or embodiments of the invention. The described embodiments are to be considered in all respects only as illustrative and not restrictive. The contents of this detailed description and the accompanying drawings do not limit the scope of the invention in any way.

Applicant has discovered that, in addition to other previously known effects, the peptide Risuteganib inhibits expression of a number of genes associated with the biosynthesis or elaboration of pro-inflammatory substances.

Risuteganib, a non-natural oligopeptide having the molecular formula C22—H39—N9—O11—S and the following structural formula:

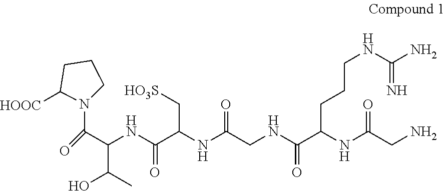 [SMILES: CC(O)C(NC(=O)C(CS(=O)(=O)O)NC(=O)CNC(=O)C(CCCNC(=N)N)NC(=O)CN)C(=O)N1CCCC1C(=O)O]

Risuteganib has also been referred to by other names, nomenclatures and designations, including: ALG-1001; Glycyl-L-arginylglycyl-3-sulfo-L-alanyl-L-threonyl-L-proline; Arg-Gly-NH—CH (CH₂—SO₃H) COOH; and Luminate® (Allegro Ophthalmics, LLC, San Juan Capistrano, CA).

Risuteganib is an anti-integrin which targets a number of integrins upstream in the oxidative stress pathway, thereby downregulating multiple angiogenic and inflammatory processes, including those associated with hypoxia and oxidative stress. Risuteganib has also been shown to improve mitochondrial function. These previously-known effects of Risuteganib and others are described in U.S. Pat. Nos. 9,018,352; 9,872,886; 9,896,480, 10,307,460; 10,639,347 and 10,590,166 and in United States Patent Application Publication Nos. US/2020/0354402; US/2020/0392181; US/2021/0002328 and US/2021/0085749 as well as co-pending U.S. patent application Ser. No. 16/854,818 entitled Compositions and Methods Useable for Treatment of Dry Eye filed Apr. 21, 2020; 16/938,758 entitled Peptides for Treating Non-Exudative Macular Degeneration And Other Disorders Of The Eye filed Jul. 24, 2020 and U.S. patent application Ser. No. 17/193832 entitled Treatments for Improving or Lessening Impairment of Mitochondrial Function filed Mar. 5, 2021, the entire disclosure of each such patent and patent application being expressly incorporated herein by reference.

Described below are studies showing that Risuteganib (ALG-1001) causes down-regulation of pro-inflammatory genes.

A: Downregulation of TNF-a pathway, immune response and leukocyte integrin genes in primary human immune cells:

The outcome of this study establishes that risuteganib effectively down-regulates TNF-a pathway, immune response and leukocyte integrin genes thereby elucidating previously unknown therapeutic uses for Risuteganib and related peptides as described below.

Purpose: In this study, RNA-seq was used to unbiasedly identify the genes regulated by risuteganib exposure in primary human immune cells. Analysis of the specific subset of genes regulated by risuteganib enables identification of biological processes and pathways modulated by the oligopeptide

Methods: Primary human immune cells (PBMC) were obtained from a single human donor. The cells were separated into two groups: vehicle or 100 μM risuteganib treatment for 8 hours. After exposure, cells were collected and their total RNA isolated for RNA-seq. The generated reads were then aligned to human reference genome/transcriptome and gene expression quantified for differential expression analysis and fold change calculation. The list of regulated genes was then used to identify biological processes and pathways that are regulated after Risuteganib exposure compared to vehicle control.

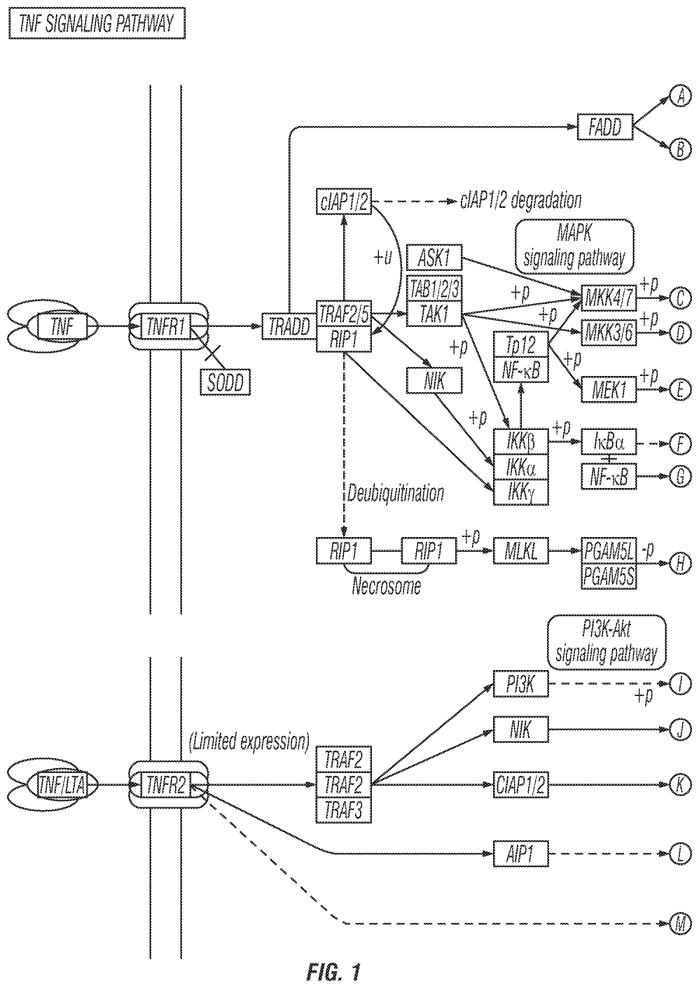

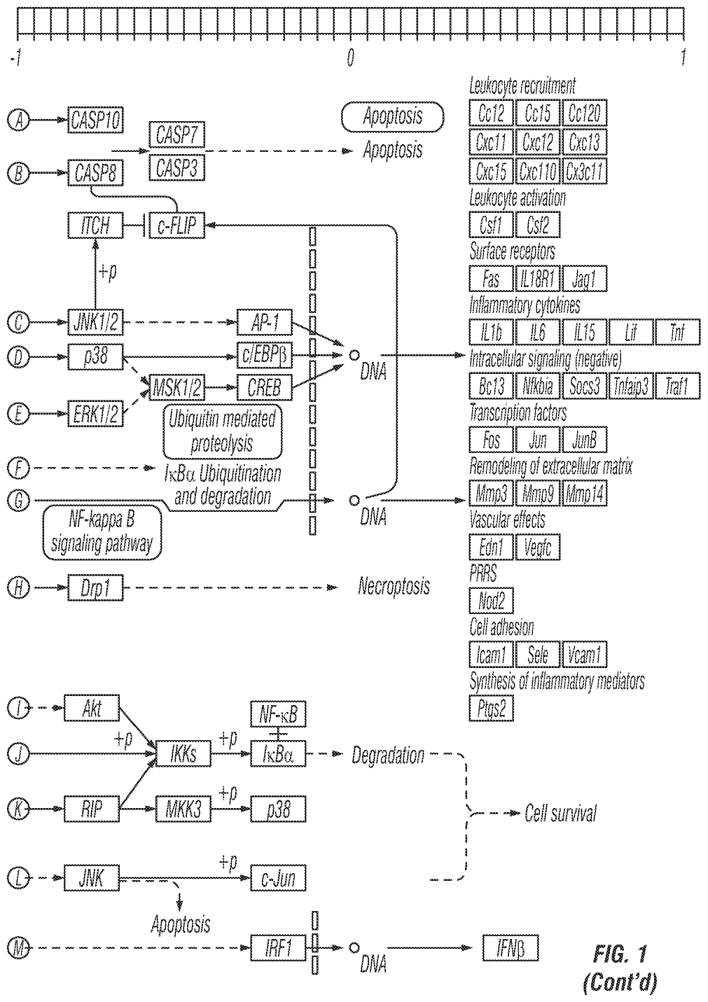

Results/Discussion: FIG. 1 shows a summary of transcriptome analysis showing that pro-inflammatory genes are selectively down-regulated after risuteganib treatment. Pathway analysis found many genes associated with TNF-a pro-inflammatory pathway are down-regulated (adjusted p-value=6.22E-3). Where indicated on FIG. 1, genes in the pathway were down-regulated. Since TNF-a pathway activation is implicated in promoting a cytotoxic inflammatory condition, Risuteganib may mediate a reduction of ocular inflammation through suppression of this pathway. In addition, APPENDIX A lists 135 immune response genes which are selectively down-regulated by risuteganib with statistical significance (adjusted p-value=5.43E-7), including TNF (pro-inflammatory cytokine), IL6 (pro-inflammatory cytokine), CCL2 (regulates inflammation), ITGAX (leukocyte-endothelial adhesion), CSF1 (regulates macrophage functions), SRC (recruits and activates immune cells), and others.

Based on the results of this study, risuteganib treatment may be used to inhibit or reduce any or all of the following:
Tumor Necrosis Factor (TNF);
Interluken 6 (IL6);
Chemokine (C-C motif) ligand 2 (CCL2);
Integrin alpha X complement component 3 receptor 4 subunit (ITGAX);
Colony stimulating factor 1 (CSF1: and/or
Src kinase (SRC).

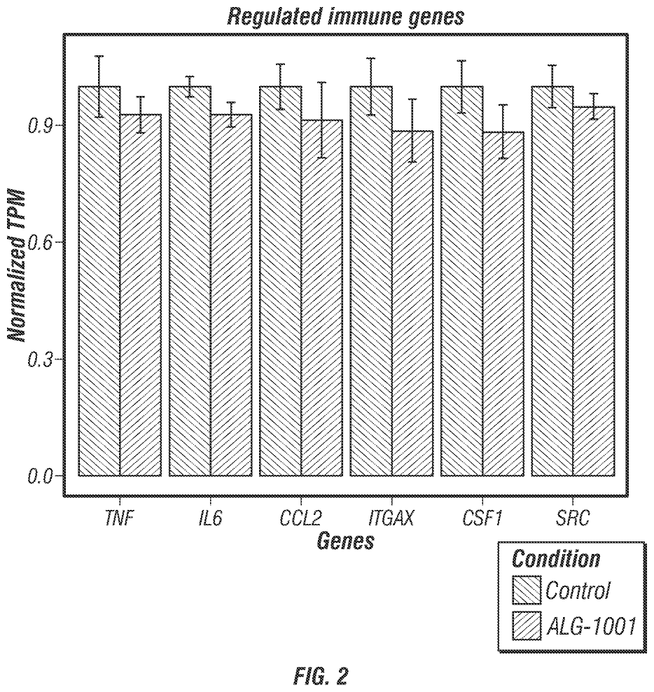

FIG. 2 and Table 1 (below) summarize the effects of risuteganib treatment on expression of above-listed genes in PMBC.

TABLE 1
The Effects of Risuteganib (ALG-1001)
on TNF-a pathway, Immune Response
and Leukocyte Integrin Genes in PMBC

| | | Mean Expression | |
|---|---|---|---|
| | | Rate | |
| | | (Normalized as | |
| Gene | Treatment | Fraction Control) | +/− S.D. |
| TNF | Control | 1 | 0.078328 |
| TNF | Risuteganib | 0.928062 | 0.044894 |
| IL6 | Control | 1 | 0.025266 |
| IL6 | Risuteganib | 0.928076 | 0.028744 |
| CCL2 | Control | 1 | 0.057688 |
| CCL2 | Risuteganib | 0.91425 | 0.096592 |
| ITGAX | Control | 1 | 0.074029 |
| ITGAX | Risuteganib | 0.886513 | 0.081571 |
| CSF1 | Control | 1 | 0.066562 |
| CSF1 | Risuteganib | 0.884149 | 0.068907 |
| SRC | Control | 1 | 0.05578 |
| SRC | Risuteganib | 0.948339 | 0.032468 |

Conclusion: Gene expression quantification shows TNF-a pathway, immune response and leukocyte integrin genes are selectively down-regulated by risuteganib in PMBC. Suppression of these pro-inflammatory and integrin genes may provide a mechanison for not only treatment or reduction of existing inflammation, but also to inhibit production of pro-inflammatory cytokines, including those which involve the TNF-a pro-inflammatory pathway, thereby providing a prophylactic or prevention strategy for avoiding predicted further inflammatory events, such as the expected elaboration of pro-inflammatory cytokines in subjects suffering from certain microbial infections.

B. Effect of Risuteganib (ALG-1001) in Human Umbilical Vein Endothelial Cells (HUVEC) Treated with Hypoxia and Cytokines

Purpose: A chronic pro-inflammatory condition is associated with many ocular diseases, including DED, AMD, and DR. During this process, tissue resident and peripheral immune cells are activated and contribute to formation of a cytotoxic inflammatory state that causes tissue damage and vision loss. In this study, RNA-seq was used to unbiasedly identify the genes regulated by risuteganib treatment in human endothelial cells exposed to hypoxia and cytokines. Analysis of the specific subset of genes regulated by risuteganib enables identification of biological processes and pathways modulated by the oligopeptide.

Methods: Human umbilical vein endothelial cells (HUVEC) were used between passage 3 to 5. All cells were grown at 3% O2 to stimulate endothelial cells into a more angiogenic state. Cells were divided into four treatment groups: control, cytokines for 48 hours, 500 uM risuteganib for 24 hours, and cytokines for 48 hours with 500 uM risuteganib for 24 hours. Cytokine treatment contains both human TNF-a and IL-1b at 1 ng/ml at final concentration. After exposure, cells were collected and their total RNA isolated for RNA-seq. The generated reads were then aligned to human reference genome/transcriptome and gene expression quantified for differential expression analysis and fold change calculation. The list of regulated genes was then used to identify biological processes and pathways that are regulated after risuteganib exposure with cytokine exposure.

Results: The Results of this study are summarized in the following Table 2, below:

TABLE 2
The Effects of Risuteganib and Risuteganib + Cytokines
(TNF-a and IL-1b)
on Gene Regulation in HUVEC

| | | Mean | |
|---|---|---|---|
| | | Expression | |
| | | Rate | |
| | | (Normalized | |
| | | as Fraction | |
| Gene | Treatment | Control) | +/− S.D. |
| CCL2 | Control | 1 | 0.092598 |
| CCL2 | Risuteganib | 0.869558 | 0.078457 |
| CCL2 | Cytokine | 51.63427 | 1.767969 |
| CCL2 | Cytokine + Risuteganib | 46.0204 | 0.644486 |
| CCL20 | Control | 1 | 0.507576 |
| CCL20 | Risuteganib | 0.632798 | 0.253193 |
| CCL20 | Cytokine | 150.401 | 7.777895 |
| CCL20 | Cytokine + Risuteganib | 128.2179 | 8.306375 |
| CXCL5 | Control | 1 | 0.512105 |
| CXCL5 | Risuteganib | 1.165991 | 0.295285 |
| CXCL5 | Cytokine | 266.0156 | 16.03164 |
| CXCL5 | Cytokine + Risuteganib | 216.416 | 13.63354 |
| CXCL6 | Control | 1 | 0.149419 |
| CXCL6 | Risuteganib | 0.969925 | 0.107909 |
| CXCL6 | Cytokine | 270.3548 | 6.952794 |
| CXCL6 | Cytokine + Risuteganib | 234.6388 | 10.90567 |
| TNFAIP6 | Control | 1 | 2.236068 |
| TNFAIP6 | Risuteganib | 0 | 0 |
| TNFAIP6 | Cytokine | 384.3986 | 32.88868 |
| TNFAIP6 | Cytokine + Risuteganib | 288.1282 | 33.69941 |
| TNFRSF11B | Control | 1 | 0.431859 |
| TNFRSF11B | Risuteganib | 0.893131 | 0.366375 |
| TNFRSF11B | Cytokine | 87.60615 | 6.256886 |
| TNFRSF11B | Cytokine + Risuteganib | 75.2753 | 5.878845 |
| SELE | Control | 1 | 0.28818 |
| SELE | Risuteganib | 0.702198 | 0.225528 |
| SELE | Cytokine | 45.17518 | 2.549873 |
| SELE | Cytokine + Risuteganib | 39.83155 | 1.585678 |
| CFH | Control | 1 | 0.096575 |
| CFH | Risuteganib | 0.970557 | 0.066617 |
| CFH | Cytokine | 1.83209 | 0.052118 |
| CFH | Cytokine + Risuteganib | 1.572239 | 0.067919 |
| IL6 | Control | 1 | 0.062932 |
| IL6 | Risuteganib | 0.99825 | 0.095579 |
| IL6 | Cytokine | 11.788 | 0.817918 |
| IL6 | Cytokine + Risuteganib | 10.55233 | 0.392164 |
| IL7R | Control | 1 | 0.082799 |
| IL7R | Risuteganib | 1.035881 | 0.122685 |
| IL7R | Cytokine | 6.91416 | 0.326639 |
| IL7R | Cytokine + Risuteganib | 5.775983 | 0.19886 |
| TNFAIP3 | Control | 1 | 0.151394 |
| TNFAIP3 | Risuteganib | 0.94598 | 0.027909 |
| TNFAIP3 | Cytokine | 12.04407 | 0.584789 |
| TNFAIP3 | Cytokine + Risuteganib | 10.74795 | 0.547217 |

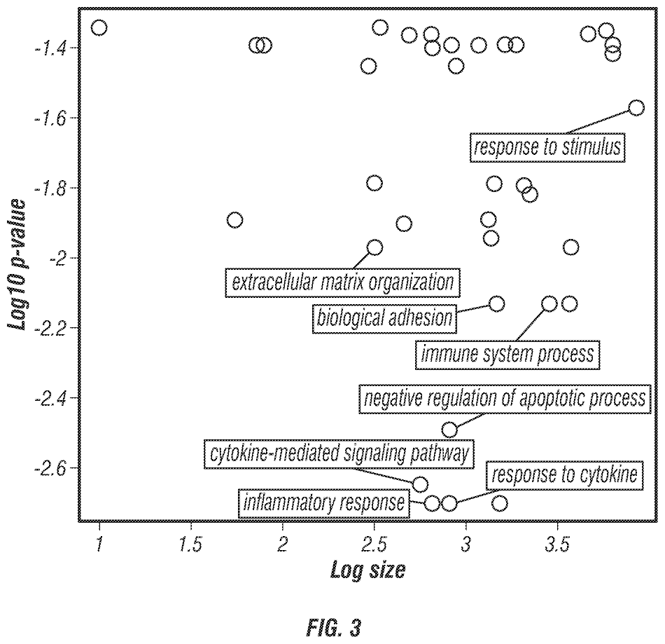

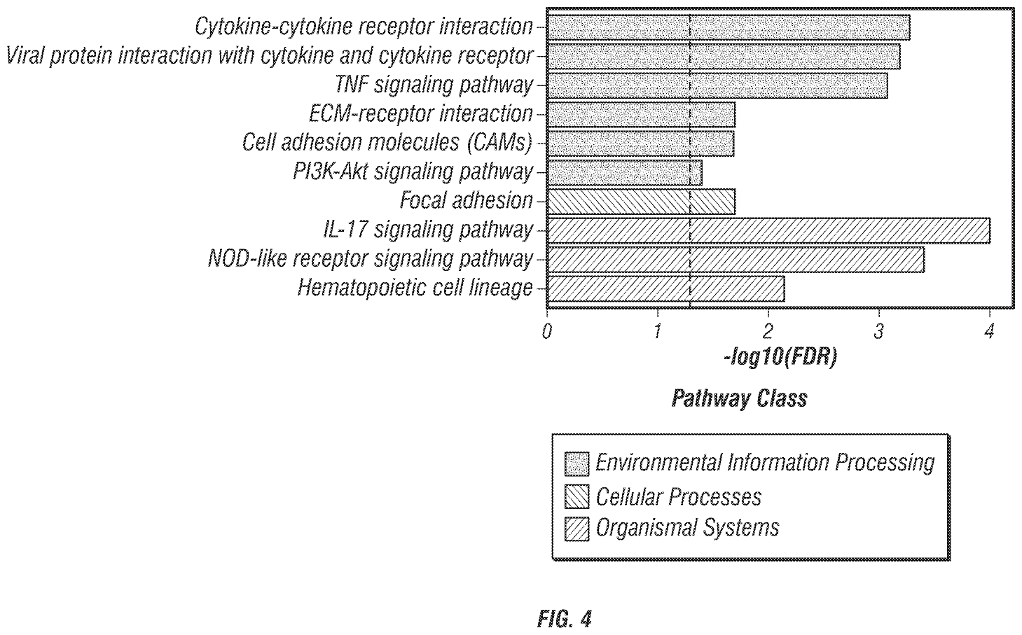

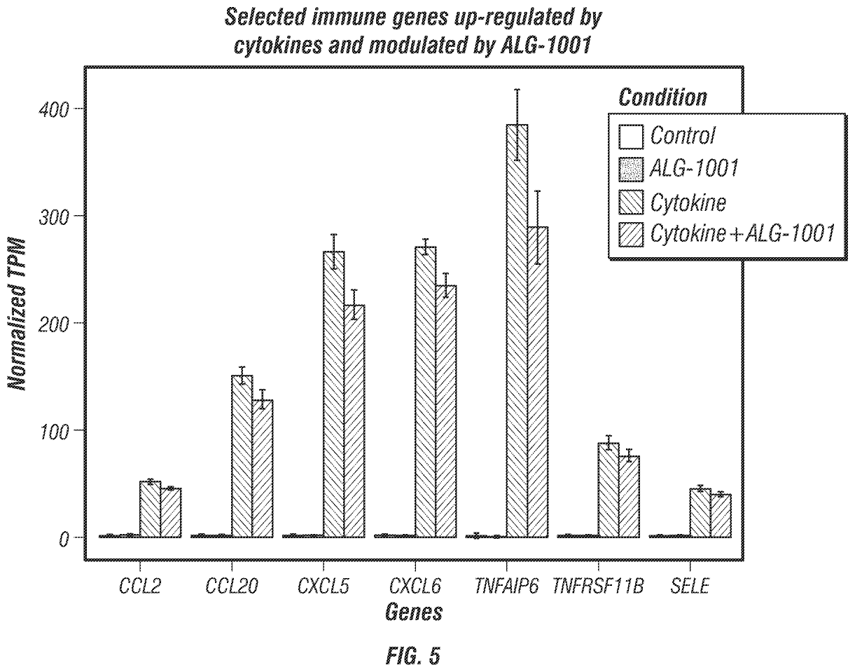

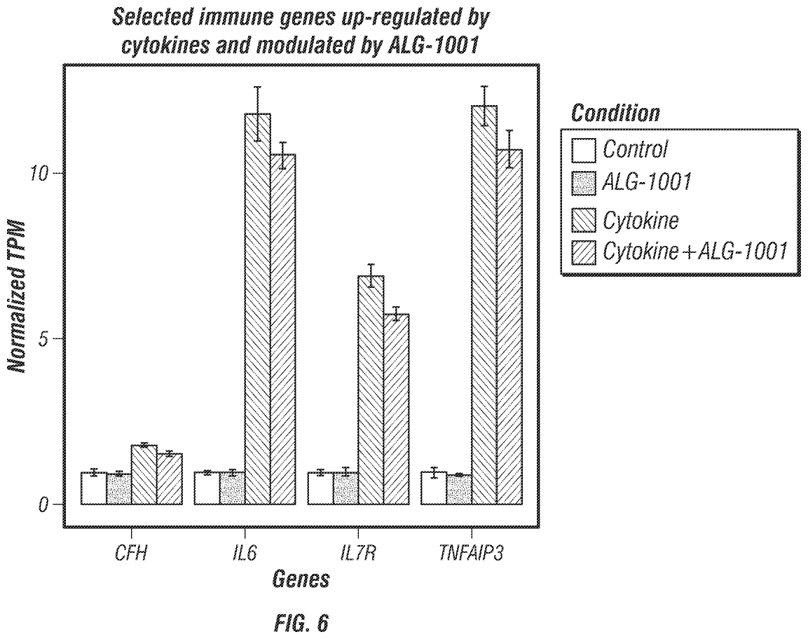

Under cytokine-activated condition, risuteganib had a moderate effect on reversing the transcriptome changes associated with cytokine treatment (Pearson's r=−0.267). 85% ( 342/403) of genes regulated by both risuteganib and cytokines are regulated in opposite directions, indicating effect of risuteganib is reverse of cytokines. As shown graphically in FIG. 3, risuteganib treatment down-regulated genes in several immune system biological processes up-regulated by cytokines, including inflammatory response, response to cytokine, and cytokine-mediated signaling pathway. As summarized in FIG. 4, pathway analysis found that ten (10) pathways were down-regulated by risuteganib, including cytokine-cytokine receptor interaction, TNF signaling pathway, and IL-17 signaling pathway, all of which are up-regulated by cytokines. Also, as seen in FIGS. 4 and 5, immune genes up-regulated by cytokines and down-regulated by risuteganib include CCL2, CCL20, CXCL5, CXCL6, TNFAIP6, TNFRSF11B, SELE, CFH, IL6, IL7R, and TNFAIP3.

Conclusion: risuteganib was found effective in suppressing the effect of TNF-a and IL-1b on the endothelial cell gene expression. Multiple inflammatory system biological processes and pathways are regulated with significance.

### Dosage and Route of Administration

The peptides disclosed herein may be administered at any effective dosages, as a single dose or multiple doses, and by any suitable routes of administration. For some systemic treatments, administration by injection or intravenous infusion may be preferable. By way of example, risuteganib has been administered to dogs by intravenous infusion at dosages of 1.0 mg/kg, 5.0 mg/kg and 8.0 mg/kg without significant toxicity or side effects. Accordingly, in at least some applications, risuteganib may be administered, for example, by intravenous infusion at a dosage ranging from 1 mg/kg to 8 mg/kg. Other dosage levels and other routes of administration may also be suitable and effective.

### Effective Peptides Other Than Risuteganib (ALG-1001)

The effects and mechanisms of action referred to in this provisional patent application are not necessarily limited to Risuteganib. Unless specified, the “other peptides”, as referenced herein may include any of the peptides described in the above-incorporated patents and applications and/or specified in this disclosure which exhibit the herein-described therapeutic effects and/or mechanisms of action. For example, peptides described in United States Patent Application Publication Nos. US/2020/0354402 and US/2020/0392181, the entire disclosures of which are expressly incorporated herein by reference, may reasonably be expected to also exhibit the herein described effects and/or mechanisms of action. Specific examples of other peptides believed to exhibit some or all of these effects or mechanisms include, but are not necessarily limited to, comprise peptides that consist of or comprise an amino acid sequence having the formula:

Y—X-Z
wherein: Y=R, H, K, Cys (acid), Gor D;
X=G, A, Cys (acid), R, G, Dor E; and
Z=Cys (acid), G, C, R, D, N or E.

Such peptides may comprise or consist of the amino acid sequences; R-G-Cys (Acid), R-R-Cys, R-CysAcid)-G, Cys (Acid)-R-G, Cys (Acid)-G-R, R-G-D, R-G-Cys (Acid). H-G-Cys (Acid), R-G-N, D-G-R, R-D-G, R-A-E, K-G-D, R-G-Cys (Acid)-G-G-G-D-G, Cyclo-{R-G-Cys (acid)-F-N-Me-V}, R-A-Cys (Acid), R-G-C, K-G-D, Cys (acid)-R-G, Cys (Acid)-G-R, Cyclo-{R-G-D-D-F-NMe-V}, H-G-Cys (acid) and salts thereof. Possible salts include but are not limited to acetate, trifluoroacetate (TFA) and hydrochloride salts.

Also, other peptides believed to exhibit some or all of these effects or mechanisms include, but are not necessarily limited to, those that consist of or comprise comprises linear or cyclic forms of Glycinyl-Arginyl-Glycinyl-Cysteic acid-Threonyl-Proline-COOH or which have the formula:

XI-R-G-Cysteic Acid-X

wherein: X and XI are selected from: Phe-Val-Ala, -Phe-Leu-Ala, -Phe-Val-Gly, -Phe-Leu-Gly, -Phe-Pro-Gly, -Phe-Pro-Ala, -Phe-Val; or from Arg, Gly, Cysteic acid, Phe, Val, Ala, Leu, Pro, Thr and salts, and any combinations of any D-isomers and L-isomers thereof.

It is to be appreciated that, although the disclosure set forth above refers to reference to certain examples or embodiments, various additions, deletions, alterations and modifications may be made to those described examples and embodiments without departing from the intended spirit and scope of the invention. For example, any elements, steps, members, components, compositions, reactants, parts or portions of one embodiment or example may be incorporated into or used with another embodiment or example, unless otherwise specified or unless doing so would render that embodiment or example unsuitable for its intended use. Also, where the steps of a method or process have been described or listed in a particular order, the order of such steps may be changed unless otherwise specified or unless doing so would render the method or process unsuitable for its intended purpose. Additionally, the elements, steps, members, components, compositions, reactants, parts or portions of any invention or example described herein may optionally exist or be utilized in the absence or substantial absence of any other element, step, member, component, composition, reactant, part or portion unless otherwise noted. All novel and reasonable additions, deletions, modifications and alterations are to be considered as equivalents of the described examples and embodiments and are to be included within the scope of the following claims.

APPENDIX A
The Effects of Risuteganib and Control on Expression
of One Hundred and Thirty-five (135) Genes in PBMC

| | | Mean | |
|---|---|---|---|
| | | Expression | |
| | | Rate | |
| | | (Normalized | |
| | | as Fraction | |
| Gene | Treatment | Control) | +/− S.D. |
| ADGRB1 | Control | 1 | 0.058372 |
| ADGRB1 | Risuteganib | 0.844405 | 0.052747 |
| OSCAR | Control | 1 | 0.099458 |
| OSCAR | ALG-1001 | 0.77197 | 0.05033 |
| FCGRT | Control | 1 | 0.046447 |
| FCGRT | Risuteganib | 0.855848 | 0.045277 |
| ITGAX | Control | 1 | 0.074029 |
| ITGAX | Risuteganib | 0.886513 | 0.081571 |
| STXBP2 | Control | 1 | 0.041161 |
| STXBP2 | Risuteganib | 0.888464 | 0.042056 |
| FOS | Control | 1 | 0.095025 |
| FOS | Risuteganib | 0.821989 | 0.059033 |
| BCL3 | Control | 1 | 0.02967 |
| BCL3 | Risuteganib | 0.891825 | 0.036628 |
| ARRB2 | Control | 1 | 0.025431 |
| ARRB2 | Risuteganib | 0.9014 | 0.043735 |
| TCIRG1 | Control | 1 | 0.04854 |
| TCIRG1 | Risuteganib | 0.891973 | 0.04185 |
| DOK3 | Control | 1 | 0.091977 |
| DOK3 | Risuteganib | 0.892942 | 0.047292 |
| MYO1G | Control | 1 | 0.043587 |
| MYO1G | Risuteganib | 0.921445 | 0.027965 |
| ANPEP | Control | 1 | 0.035974 |
| ANPEP | Risuteganib | 0.908151 | 0.030869 |
| RARA | Control | 1 | 0.049335 |
| RARA | Risuteganib | 0.892585 | 0.074439 |
| NA | Control | 1 | 0.055337 |
| NA | Risuteganib | 0.907738 | 0.044992 |
| CSF1 | Control | 1 | 0.066562 |
| CSF1 | Risuteganib | 0.884149 | 0.068907 |
| TICAM1 | Control | 1 | 0.029824 |
| TICAM1 | Risuteganib | 0.938197 | 0.023824 |
| GAA | Control | 1 | 0.050518 |
| GAA | Risuteganib | 0.912103 | 0.0355 |
| IL1RN | Control | 1 | 0.084993 |
| IL1RN | Risuteganib | 0.846887 | 0.053414 |
| ZYX | Control | 1 | 0.051378 |
| ZYX | Risuteganib | 0.913904 | 0.053527 |
| KCNN4 | Control | 1 | 0.05996 |
| KCNN4 | Risuteganib | 0.912656 | 0.036786 |
| ERCC1 | Control | 1 | 0.063176 |
| ERCC1 | Risuteganib | 0.878369 | 0.039776 |
| RAPGEF1 | Control | 1 | 0.049238 |
| RAPGEF1 | Risuteganib | 0.945892 | 0.043312 |
| IGLV2-23 | Control | 1 | 0.455588 |
| IGLV2-23 | Risuteganib | 0.45035 | 0.149072 |
| CYBA | Control | 1 | 0.051461 |
| CYBA | Risuteganib | 0.886884 | 0.042965 |
| SLC11A1 | Control | 1 | 0.099429 |
| SLC11A1 | Risuteganib | 0.88423 | 0.065205 |
| FGR | Control | 1 | 0.027032 |
| FGR | Risuteganib | 0.922169 | 0.029962 |
| LTB4R | Control | 1 | 0.124416 |
| LTB4R | Risuteganib | 0.833961 | 0.044173 |
| TRPM2 | Control | 1 | 0.075297 |
| TRPM2 | Risuteganib | 0.939079 | 0.018547 |
| GRN | Control | 1 | 0.02934 |
| GRN | Risuteganib | 0.92211 | 0.054538 |
| CCL3L1 | Control | 1 | 0.062259 |
| CCL3L1 | Risuteganib | 0.8739 | 0.054467 |
| SRC | Control | 1 | 0.05578 |
| SRC | Risuteganib | 0.948339 | 0.032468 |
| NFKBID | Control | 1 | 0.082527 |
| NFKBID | Risuteganib | 0.920335 | 0.051186 |
| PI3 | Control | 1 | 0.088975 |
| PI3 | Risuteganib | 0.915827 | 0.01149 |
| IFI30 | Control | 1 | 0.056415 |
| IFI30 | Risuteganib | 0.831835 | 0.12747 |
| PLD2 | Control | 1 | 0.097727 |
| PLD2 | Risuteganib | 0.921536 | 0.075338 |
| CCL22 | Control | 1 | 0.084677 |
| CCL22 | Risuteganib | 0.919743 | 0.028328 |
| HIST2H2BE | Control | 1 | 0.097352 |
| HIST2H2BE | Risuteganib | 0.852665 | 0.051382 |
| CDC37 | Control | 1 | 0.032816 |
| CDC37 | Risuteganib | 0.934954 | 0.037478 |
| TNFRSF14 | Control | 1 | 0.096816 |
| TNFRSF14 | Risuteganib | 0.923904 | 0.059025 |
| IL6 | Control | 1 | 0.025266 |
| IL6 | Risuteganib | 0.928076 | 0.028744 |
| KCNAB2 | Control | 1 | 0.055173 |
| KCNAB2 | Risuteganib | 0.95884 | 0.057164 |
| HLX | Control | 1 | 0.087172 |
| HLX | Risuteganib | 0.908398 | 0.030569 |
| IRF5 | Control | 1 | 0.048416 |
| IRF5 | Risuteganib | 0.92929 | 0.042144 |
| STK10 | Control | 1 | 0.041614 |
| STK10 | Risuteganib | 0.96059 | 0.034657 |
| TNF | Control | 1 | 0.078328 |
| TNF | Risuteganib | 0.928062 | 0.044894 |
| GALNS | Control | 1 | 0.04531 |
| GALNS | Risuteganib | 0.935622 | 0.067001 |
| SOCS1 | Control | 1 | 0.069235 |
| SOCS1 | Risuteganib | 0.928932 | 0.055177 |
| NFKB2 | Control | 1 | 0.043121 |
| NFKB2 | Risuteganib | 0.967454 | 0.039104 |
| BAG6 | Control | 1 | 0.023212 |
| BAG6 | Risuteganib | 0.952246 | 0.04458 |
| CEBPB | Control | 1 | 0.10748 |
| CEBPB | Risuteganib | 0.875196 | 0.037858 |
| TOM1 | Control | 1 | 0.038895 |
| TOM1 | Risuteganib | 0.921097 | 0.033928 |
| CXCL2 | Control | 1 | 0.155699 |
| CXCL2 | Risuteganib | 0.85238 | 0.017093 |
| STX4 | Control | 1 | 0.014365 |
| STX4 | Risuteganib | 0.948985 | 0.037898 |
| UNC13D | Control | 1 | 0.07147 |
| UNC13D | Risuteganib | 0.957742 | 0.036487 |
| CYB5R3 | Control | 1 | 0.037882 |
| CYB5R3 | Risuteganib | 0.956411 | 0.043122 |
| LILRB3 | Control | 1 | 0.051815 |
| LILRB3 | Risuteganib | 0.939631 | 0.0412 |
| FCER2 | Control | 1 | 0.267912 |
| FCER2 | Risuteganib | 0.768577 | 0.048754 |
| SUPT6H | Control | 1 | 0.067635 |
| SUPT6H | Risuteganib | 0.956802 | 0.045627 |
| CTSA | Control | 1 | 0.036818 |
| CTSA | Risuteganib | 0.940959 | 0.052579 |
| HLA-DPB1 | Control | 1 | 0.046857 |
| HLA-DPB1 | Risuteganib | 0.929475 | 0.046115 |
| MAP2K7 | Control | 1 | 0.043733 |
| MAP2K7 | Risuteganib | 0.94205 | 0.069067 |
| ARSA | Control | 1 | 0.075754 |
| ARSA | Risuteganib | 0.923934 | 0.104336 |
| CXCL1 | Control | 1 | 0.05192 |
| CXCL1 | Risuteganib | 0.92163 | 0.024911 |
| ATP6V0A1 | Control | 1 | 0.068929 |
| ATP6V0A1 | Risuteganib | 0.943167 | 0.048355 |
| TYROBP | Control | 1 | 0.038748 |
| TYROBP | Risuteganib | 0.932938 | 0.040897 |
| IGLC2 | Control | 1 | 0.130993 |
| IGLC2 | Risuteganib | 0.846731 | 0.111736 |
| OSM | Control | 1 | 0.176878 |
| OSM | Risuteganib | 0.830581 | 0.0824 |
| MCOLN1 | Control | 1 | 0.058736 |
| MCOLN1 | Risuteganib | 0.9403 | 0.04619 |
| ALDH3B1 | Control | 1 | 0.072006 |
| ALDH3B1 | Risuteganib | 0.891177 | 0.091234 |
| PRKCD | Control | 1 | 0.040014 |
| PRKCD | Risuteganib | 0.963149 | 0.023815 |
| PLAUR | Control | 1 | 0.063841 |
| PLAUR | Risuteganib | 0.910427 | 0.049279 |
| PSTPIP1 | Control | 1 | 0.091299 |
| PSTPIP1 | Risuteganib | 0.940931 | 0.057366 |
| TFE3 | Control | 1 | 0.04315 |
| TFE3 | Risuteganib | 0.973128 | 0.040434 |
| LIF | Control | 1 | 0.281398 |
| LIF | Risuteganib | 0.793918 | 0.022479 |
| RNF19B | Control | 1 | 0.079259 |
| RNF19B | Risuteganib | 0.946929 | 0.052555 |
| NCF4 | Control | 1 | 0.061551 |
| NCF4 | Risuteganib | 0.943299 | 0.075669 |
| LIMK1 | Control | 1 | 0.066869 |
| LIMK1 | Risuteganib | 0.928497 | 0.041716 |
| BRI3 | Control | 1 | 0.044619 |
| BRI3 | Risuteganib | 0.939683 | 0.041781 |
| OTUB1 | Control | 1 | 0.043505 |
| OTUB1 | Risuteganib | 0.94449 | 0.035608 |
| DGAT1 | Control | 1 | 0.072854 |
| DGAT1 | Risuteganib | 0.930801 | 0.085853 |
| MMP25 | Control | 1 | 0.118261 |
| MMP25 | Risuteganib | 0.895877 | 0.069705 |
| CKAP4 | Control | 1 | 0.067979 |
| CKAP4 | Risuteganib | 0.913339 | 0.050241 |
| RAB5C | Control | 1 | 0.058242 |
| RAB5C | Risuteganib | 0.924608 | 0.051347 |
| TNFSF9 | Control | 1 | 0.062125 |
| TNFSF9 | Risuteganib | 0.841613 | 0.099839 |
| MMP9 | Control | 1 | 0.107033 |
| MMP9 | Risuteganib | 0.912519 | 0.075715 |
| NBEAL2 | Control | 1 | 0.118057 |
| NBEAL2 | Risuteganib | 0.943152 | 0.062258 |
| SLC26A6 | Control | 1 | 0.038814 |
| SLC26A6 | Risuteganib | 0.945244 | 0.067688 |
| GPR84 | Control | 1 | 0.046945 |
| GPR84 | Risuteganib | 0.923137 | 0.087878 |
| TREM1 | Control | 1 | 0.04516 |
| TREM1 | Risuteganib | 0.908418 | 0.084485 |
| NPLOC4 | Control | 1 | 0.032732 |
| NPLOC4 | Risuteganib | 0.974751 | 0.036537 |
| MCEMP1 | Control | 1 | 0.124267 |
| MCEMP1 | Risuteganib | 0.860031 | 0.089296 |
| TNFRSF4 | Control | 1 | 0.027581 |
| TNFRSF4 | Risuteganib | 0.942656 | 0.03893 |
| RHOG | Control | 1 | 0.018798 |
| RHOG | Risuteganib | 0.959166 | 0.01943 |
| MUCL1 | Control | 1 | 0.268305 |
| MUCL1 | Risuteganib | 0.686558 | 0.175156 |
| AGER | Control | 1 | 0.067781 |
| AGER | Risuteganib | 0.913557 | 0.107937 |
| GLA | Control | 1 | 0.044517 |
| GLA | Risuteganib | 0.963933 | 0.026448 |
| CCL4L2 | Control | 1 | 0.044856 |
| CCL4L2 | Risuteganib | 0.938898 | 0.040848 |
| HLA-DQB1 | Control | 1 | 0.048138 |
| HLA-DQB1 | Risuteganib | 0.955383 | 0.050298 |
| LILRB4 | Control | 1 | 0.09098 |
| LILRB4 | Risuteganib | 0.896959 | 0.100792 |
| FCER1G | Control | 1 | 0.027343 |
| FCER1G | Risuteganib | 0.928856 | 0.066972 |
| CXCL16 | Control | 1 | 0.028739 |
| CXCL16 | Risuteganib | 0.932613 | 0.074639 |
| CD14 | Control | 1 | 0.138012 |
| CD14 | Risuteganib | 0.889879 | 0.044699 |
| DYSF | Control | 1 | 0.160099 |
| DYSF | Risuteganib | 0.858715 | 0.089913 |
| SIRPA | Control | 1 | 0.029956 |
| SIRPA | Risuteganib | 0.965712 | 0.030979 |
| S100A11 | Control | 1 | 0.038815 |
| S100A11 | Risuteganib | 0.949181 | 0.031033 |
| CLCF1 | Control | 1 | 0.120667 |
| CLCF1 | Risuteganib | 0.897544 | 0.039292 |
| CSF3 | Control | 1 | 0.157957 |
| CSF3 | Risuteganib | 0.861451 | 0.075777 |
| PILRA | Control | 1 | 0.037922 |
| PILRA | Risuteganib | 0.945718 | 0.056582 |
| CTSD | Control | 1 | 0.069531 |
| CTSD | Risuteganib | 0.917261 | 0.054053 |
| IGHD | Control | 1 | 0.139459 |
| IGHD | Risuteganib | 0.873078 | 0.10457 |
| NRROS | Control | 1 | 0.041292 |
| NRROS | Risuteganib | 0.920192 | 0.068063 |
| CD300A | Control | 1 | 0.050692 |
| CD300A | Risuteganib | 0.913688 | 0.087832 |
| IL4R | Control | 1 | 0.037254 |
| IL4R | Risuteganib | 0.97475 | 0.055306 |
| GPX1 | Control | 1 | 0.055589 |
| GPX1 | Risuteganib | 0.925449 | 0.055385 |
| CCL4 | Control | 1 | 0.04192 |
| CCL4 | Risuteganib | 0.960459 | 0.023396 |
| TNFRSF1B | Control | 1 | 0.046065 |
| TNFRSF1B | Risuteganib | 0.960367 | 0.046545 |
| TYK2 | Control | 1 | 0.050676 |
| TYK2 | Risuteganib | 0.971082 | 0.089442 |
| SH2B2 | Control | 1 | 0.088633 |
| SH2B2 | Risuteganib | 0.878952 | 0.129356 |
| MT2A | Control | 1 | 0.086044 |
| MT2A | Risuteganib | 0.921581 | 0.034426 |
| TRAF3IP2 | Control | 1 | 0.058812 |
| TRAF3IP2 | Risuteganib | 0.947174 | 0.027807 |
| LRP1 | Control | 1 | 0.092433 |
| LRP1 | Risuteganib | 0.940487 | 0.053505 |
| HEXIM1 | Control | 1 | 0.04086 |
| HEXIM1 | Risuteganib | 0.965347 | 0.033706 |
| PFKL | Control | 1 | 0.079352 |
| PFKL | Risuteganib | 0.980421 | 0.044244 |
| ADGRG3 | Control | 1 | 0.201159 |
| ADGRG3 | Risuteganib | 0.838064 | 0.099337 |
| CCL2 | Control | 1 | 0.057688 |
| CCL2 | Risuteganib | 0.91425 | 0.096592 |
| ZP3 | Control | 1 | 0.138212 |
| ZP3 | Risuteganib | 0.84915 | 0.070134 |
| NFAM1 | Control | 1 | 0.146323 |
| NFAM1 | Risuteganib | 0.894765 | 0.077453 |
| CSTB | Control | 1 | 0.039313 |
| CSTB | Risuteganib | 0.964884 | 0.027467 |
| ITGAL | Control | 1 | 0.041587 |
| ITGAL | Risuteganib | 0.984212 | 0.031086 |
| PDXK | Control | 1 | 0.051707 |
| PDXK | Risuteganib | 0.973002 | 0.0151 |
| RAC2 | Control | 1 | 0.028325 |
| RAC2 | Risuteganib | 0.976281 | 0.027917 |
| NECTIN2 | Control | 1 | 0.052502 |
| NECTIN2 | Risuteganib | 0.94786 | 0.060706 |
| TNFRSF13B | Control | 1 | 0.174763 |
| TNFRSF13B | Risuteganib | 0.850784 | 0.102807 |
| PRAM1 | Control | 1 | 0.15564 |
| PRAM1 | Risuteganib | 0.866123 | 0.082739 |
| ADGRE2 | Control | 1 | 0.11165 |
| ADGRE2 | Risuteganib | 0.923001 | 0.089589 |
| AKIRIN2 | Control | 1 | 0.044164 |
| AKIRIN2 | Risuteganib | 0.969677 | 0.031246 |
| LFNG | Control | 1 | 0.037702 |
| LFNG | Risuteganib | 0.973438 | 0.033542 |
| CD63 | Control | 1 | 0.031622 |
| CD63 | Risuteganib | 0.948686 | 0.044778 |
| SP2 | Control | 1 | 0.057673 |
| SP2 | Risuteganib | 0.961341 | 0.064253 |
| ITGAM | Control | 1 | 0.06632 |
| ITGAM | Risuteganib | 0.947304 | 0.07723 |
| ZC3H12A | Control | 1 | 0.042122 |
| ZC3H12A | Risuteganib | 0.975673 | 0.028964 |
| CYP27B1 | Control | 1 | 0.0837 |
| CYP27B1 | Risuteganib | 0.862336 | 0.153154 |
| QSOX1 | Control | 1 | 0.060581 |
| QSOX1 | Risuteganib | 0.935494 | 0.055073 |
| ORAI1 | Control | 1 | 0.045546 |
| ORAI1 | Risuteganib | 0.971417 | 0.062968 |
| LTBR | Control | 1 | 0.042272 |
| LTBR | Risuteganib | 0.964366 | 0.058224 |
| DNAJC5 | Control | 1 | 0.036475 |
| DNAJC5 | Risuteganib | 0.969041 | 0.019835 |
| RELB | Control | 1 | 0.038465 |
| RELB | Risuteganib | 0.964659 | 0.028778 |
| HRAS | Control | 1 | 0.048436 |
| HRAS | Risuteganib | 0.923784 | 0.065208 |
| MAN2B1 | Control | 1 | 0.069293 |
| MAN2B1 | Risuteganib | 0.970587 | 0.045087 |
| FABP5 | Control | 1 | 0.0887 |
| FABP5 | Risuteganib | 0.871322 | 0.124053 |
| ADAM8 | Control | 1 | 0.064273 |
| ADAM8 | Risuteganib | 0.970756 | 0.031124 |

1. A method for inhibiting a pro-inflammatory cytokines in a subject in need thereof, said method comprising the step:
of administering to the subject a therapeutically effective amount risuteganib.

2. A method according to claim 1 carried out for preventing or reducing severity of hypercytokinemia, cytokine release syndrome or cytokine storm in a subject in need thereof.

3. A method according to claim 1 carried out for inhibiting one or more endogenous substance selected from: tumor necrosis factor (TNF), interluken 6 (IL6), CCL2, ITGAX, CSF1, and SRC, in a in a subject in need thereof.

4. A method according to claim 1 performed in the treatment of an infectious disease.

5. A method according to claim 4 wherein the infectious disease comprises: an influenza, mycobacterial infection, viral infection, coronavirus infection, SARS-CoV-2 (COVID-19) infection or pneumonia.

6. A method according to any of claim 1 performed in treatment of an autoimmune disorder.

7. A method according to claim 6 wherein the autoimmune disorder comprises: rheumatoid arthritis (RA), osteoarthritis, juvenile arthritis, psoriatic arthritis, plaque psoriasis, ankylosing spondylitis, ulcerative colitis (UC), and Crohn's disease.

8. A method according to claim 1 performed to mitigate inflammation or cytokine-mediated tissue impairment or damage in the subject following diagnosis of a disease or disorder selected from; an influenza, mycobacterial infection, viral infection, coronavirus infection, SARS-COV-2 (COVID-19) infection, pneumonia, an autoimmune disorder, rheumatoid arthritis (RA), osteoarthritis, juvenile arthritis, psoriatic arthritis, plaque psoriasis, ankylosing spondylitis, ulcerative colitis (UC), or Crohn's disease.

9. (canceled)

10. A method according to claim 1 performed to mitigate inflammation or cytokine-mediated tissue impairment or damage in a human or non-human animal subject who has been diagnosed with a disease or disorder selected from; an influenza, mycobacterial infection, viral infection, coronavirus infection, SARS-COV-2 (COVID-19) infection, pneumonia, an autoimmune disorder, rheumatoid arthritis (RA), osteoarthritis, juvenile arthritis, psoriatic arthritis, plaque psoriasis, ankylosing spondylitis, ulcerative colitis (UC), or Crohn's disease.

11-15. (canceled)
